(19) United States
(12) Patent Application Publication
(10) Pub. No.: US 2015/0112234 A1
(43) Pub. Date: Apr. 23, 2015

(54) DEVICES, SYSTEMS, AND METHODS FOR THE SELECTIVE POSITIONING OF AN INTRAVASCULAR ULTRASOUND NEUROMODULATION DEVICE

(71) Applicant: Medtronic Ardian Luxembourg S.a.r.I, Luxembourg (LU)

(72) Inventors: Gerry McCaffrey, Galway (IE); Barry O'Connell, Galway (IE)

(21) Appl. No.: 14/058,096

(22) Filed: Oct. 18, 2013

(51) Int. Cl. A61N 7/02

(52) U.S. Cl. CPC A61N 7/02; A61N 2007/0026
USPC 601/3

(57) ABSTRACT

Devices, systems, and methods for the selective positioning of an intravascular ultrasound neuromodulation device are disclosed herein. One aspect of the present technology is directed to positioning systems for focused ultrasound devices. Some embodiments, for example, are directed to dual-balloon positioning systems. Such systems can include, for example, an elongated shaft and a therapeutic assembly and a balloon assembly carried by a distal portion of the elongated shaft. The therapeutic assembly is configured for delivery within a blood vessel. The balloon assembly can include a first balloon and a second balloon circumferentially offset from the first balloon about the elongated shaft. The first and second balloons can be selectively inflated to position an ultrasound transducer of the therapeutic assembly at a precise location within the blood vessel.

### TECHNICAL FIELD

The present technology relates generally to ultrasound in the context of neuromodulation devices, systems, and methods. Some embodiments, for example, are directed to an ultrasound system having two or more balloons configured to provide precise spatial positioning of an ultrasound transducer within a blood vessel.

### BACKGROUND

The sympathetic nervous system (SNS) is a primarily involuntary bodily control system typically associated with stress responses. SNS fibers that innervate tissue are present in almost every organ system of the human body and can affect characteristics such as pupil diameter, gut motility, and urinary output. Such regulation can have adaptive utility in maintaining homeostasis or preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (“NE”) spillover rates in patients with essential hypertension.

Sympathetic nerves innervating the kidneys terminate in the blood vessels, the juxtaglomerular apparatus, and the renal tubules. Stimulation of the renal sympathetic nerves can cause increased renin release, increased sodium (Na⁺) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate that result from renal sympathetic efferent stimulation are likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology.

FIG. 1 is a partially-schematic perspective view illustrating a renal neuromodulation system including a catheter configured in accordance with an embodiment of the present technology.

FIG. 2A is a side view of the distal portion of the catheter in a deployed state (e.g., expanded or inflated configuration) positioned in a human blood vessel in accordance with an embodiment of the present technology.

FIG. 2B1 is an isolated, enlarged view of the ultrasound transducer of FIG. 2A configured in accordance with embodiments of the present technology.

FIG. 2B2 is an isolated, enlarged view of an ultrasound transducer configured in accordance with embodiments of the present technology.

FIG. 2C is a cross-sectional end view of the distal portion of the catheter in FIG. 2A taken along the line 2C-2C.

FIG. 2D is a cross-sectional end view of the elongated shaft in FIG. 2A taken along the line 2D-2D.

FIG. 2E is a side view of the distal portion of the catheter in a deployed state with the balloon assembly distal to the therapeutic assembly in accordance with an embodiment of the present technology.

FIG. 2F is a side view of the distal portion of the catheter in a deployed state with the balloon assembly over the therapeutic assembly in accordance with an embodiment of the present technology.

FIGS. 3A-3B are schematic representations of a transducer within vessels of differing diameters for illustrative purposes only.

FIG. 4A is a side view of the distal portion of a catheter having an irrigation port in a deployed state in accordance with an embodiment of the present technology.

FIG. 4B is a cross-sectional end view of the distal portion of the catheter in FIG. 4A.

FIG. 5A is a side view of the distal portion of a catheter in a deployed state having a first balloon assembly and a second balloon assembly in accordance with an embodiment of the present technology.

FIG. 5B is a cross-sectional end view of the distal portion of the catheter in FIG. 5A taken along the line 5B-5B.

FIG. 5C is a cross-sectional end view of a distal portion of a catheter having a balloon assembly with three balloons in accordance with another embodiment of the present technology.

FIG. 6 is a partially cross-sectional anatomical front view illustrating advancing the catheter shown in FIG. 1 along an intravascular path in accordance with an embodiment of the present technology.

FIG. 7 is a side view of the distal portion of the catheter in a delivery state (e.g., low-profile) positioned in a human blood vessel in accordance with an embodiment of the present technology.

FIG. 8A is a side view of the distal portion of the catheter in a deployed state positioned in a human blood vessel in accordance with an embodiment of the present technology.

FIG. 8B is a cross-sectional end view of the distal portion of the catheter in FIG. 8A.

FIG. 9A is a side view of the distal portion of the catheter in a deployed state positioned in a human blood vessel, wherein the second balloon is inflated to a greater volume than the first balloon in accordance with an embodiment of the present technology.

FIG. 9B is a cross-sectional end view of the distal portion of the catheter in FIG. 9A.

FIG. 10A is a side view of the distal portion of the catheter in a deployed state positioned in a relatively narrow human blood vessel in accordance with an embodiment of the present technology.

FIG. 10B is a side view of the distal portion of the catheter in a deployed state positioned in a relatively wide human blood vessel in accordance with an embodiment of the present technology.

FIG. 11 is a top view of a portion of a balloon having a plurality of pressure sensors configured in accordance with an embodiment of the present technology.

FIG. 12 is a conceptual diagram illustrating the sympathetic nervous system and how the brain communicates with the body via the sympathetic nervous system.

FIG. 13 is an enlarged anatomical view illustrating nerves innervating a left kidney to form a renal plexus surrounding a left renal artery.

FIGS. 14A and 14B are anatomical and conceptual views, respectively, illustrating a human body including a brain and kidneys and neural efferent and afferent communication between the brain and kidneys.

FIGS. 15A and 15B are anatomic views illustrating, respectively, an arterial vasculature and a venous vasculature of a human.

### DETAILED DESCRIPTION

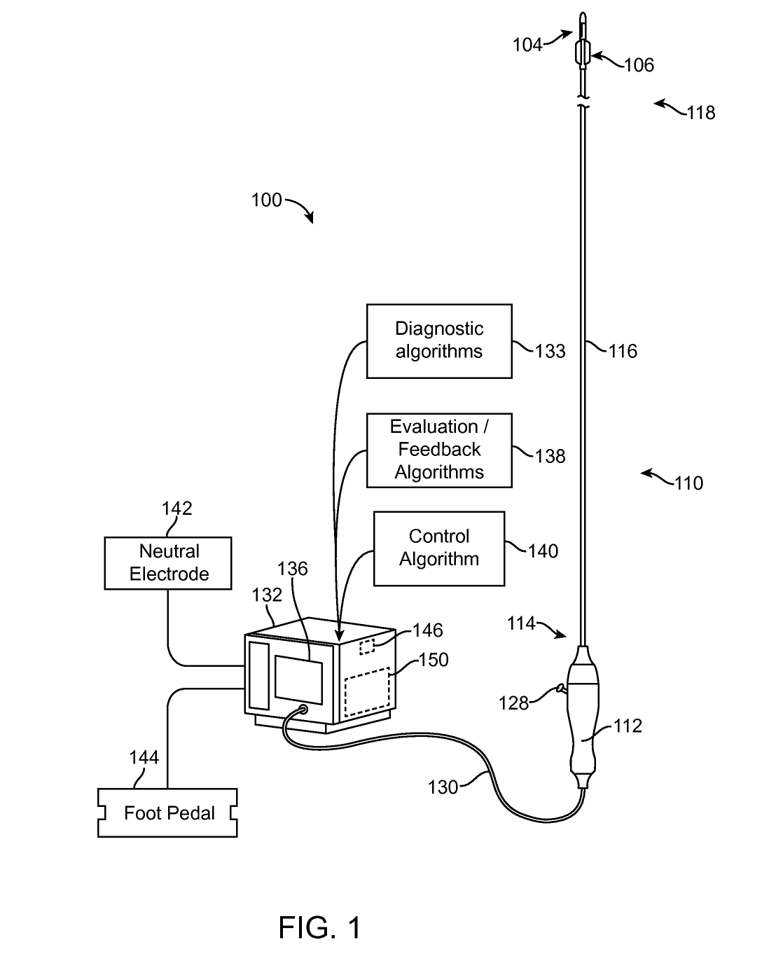

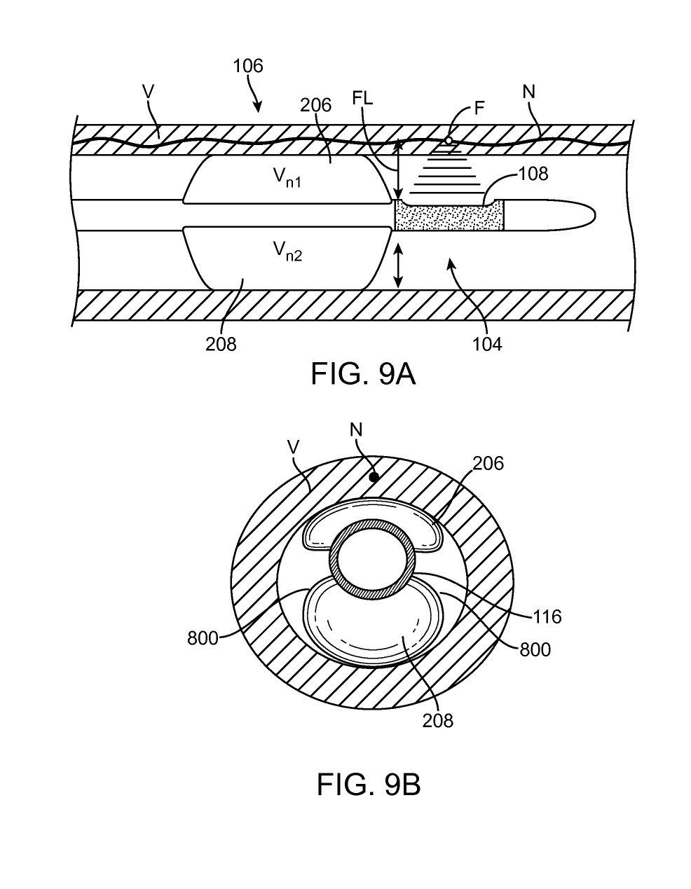

The present technology is directed to ultrasound in the context of neuromodulation devices, systems, and methods. Several embodiments of the present technology, for example, are directed to catheters, catheter systems, and methods having two or more balloons configured for positioning therapeutic ultrasound transducers within a vessel. Specific details of several embodiments of the technology are described below with reference to FIGS. 1-15B. Although many of the embodiments are described below with respect to systems, devices, and methods for positioning therapeutic ultrasound devices for neuromodulation, other applications (e.g., positioning systems for other interventional devices such as RF ablation catheters, cryo-ablation catheters, etc. or are alternatively related to neuromodulation of other peripheral nerves, treatments other than neuromodulation, etc.) and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-15B.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

### I. NEUROMODULATION

Neuromodulation is the partial or complete incapacitation or other effective disruption of nerves innervating, for example, an organ. As an example, renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and, in particular, conditions associated with central sympathetic overstimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, osteoporosis, and sudden death, among others. The reduction of afferent neural signals typically contributes to the systemic reduction of sympathetic tone/drive, and renal neuromodulation is expected to be useful in treating several conditions associated with systemic sympathetic overactivity or hyperactivity. Renal neuromodulation can potentially benefit a variety of organs and bodily structures innervated by sympathetic nerves.

Thermal effects can include both thermal ablation and non-ablative thermal alteration or damage (e.g., via sustained heating and/or resistive heating) to partially or completely disrupt the ability of a nerve to transmit a signal. Desired thermal heating effects, for example, may include raising the temperature of neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for ablative thermal alteration.

More specifically, exposure to thermal energy in excess of a body temperature of about 37° C., but below a temperature of about 45° C., may induce thermal alteration via moderate heating of neural fibers or of vascular structures that perfuse the target fibers. In cases where vascular structures are affected, the neural fibers may be denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures that perfuse the target fibers. In some patients, it may be desirable to achieve temperatures that thermally ablate the target neural fibers or the vascular structures, but that are less than about 90° C., or less than about 85° C., or less than about 80° C., and/or less than about 75° C. Other embodiments can include heating tissue to a variety of other suitable temperatures. Regardless of the type of heat exposure utilized to induce the thermal neuromodulation, a reduction in renal sympathetic nerve activity (RSNA) is expected.

Various techniques can be used to partially or completely incapacitate neural pathways, such as those innervating the kidneys. The purposeful application of energy (e.g., RF energy, mechanical energy, acoustic energy, electrical energy, thermal energy, etc.) to tissue and/or the purposeful removal of energy (e.g., thermal energy) from tissue can induce one or more desired thermal heating and/or cooling effects on localized regions of the tissue. The tissue, for example, can be tissue of the renal artery and adjacent regions of the renal plexus, which lay intimately within or adjacent to the adventitia of the renal artery. For example, the purposeful application and/or removal of energy can be used to achieve therapeutically effective neuromodulation along all or a portion of the renal plexus.

Ultrasound therapy involves delivering ultrasound energy to localized regions of tissue from externally (non-invasive) or internally (minimally-invasive) located transducers. When ultrasound energy is applied to anatomical tissue, significant physiological effects can be produced in the anatomical tissue resulting from thermal and/or mechanical changes or effects in the tissue. Transducers emit ultrasound energy to a known focal point to produce such thermal effects at desired anatomical locations. Various design features of a particular transducer determine and fix the focal point of that transducer, such as, for example, the size, shape, and/or material of the transducer. A challenge in the application of such ultrasound transducers is that in order for the focal point to coincide with the desired anatomical location, the device carrying the transducer must be correctly positioned within the vessel. If the device is incorrectly positioned so that the focal point does not correspond with the desired anatomical location, the targeted tissue may not receive sufficient ultrasound energy to affect therapeutic treatment. Moreover, incorrect positioning can result in non-target tissue damage. For example, if the transducer is incorrectly positioned, the focal point may lie on non-target tissue and/or non-target tissue may be damaged. To address these needs, the present technology provides several embodiments of devices, systems, and methods to position an ultrasound transducer at a desired location within the blood vessel such that the ultrasound waves from the ultrasound transducer are selectively focused on a target depth where neural fibers are likely to be located within the blood vessel wall.

### II. SELECTED EMBODIMENTS OF NEUROMODULATION SYSTEMS

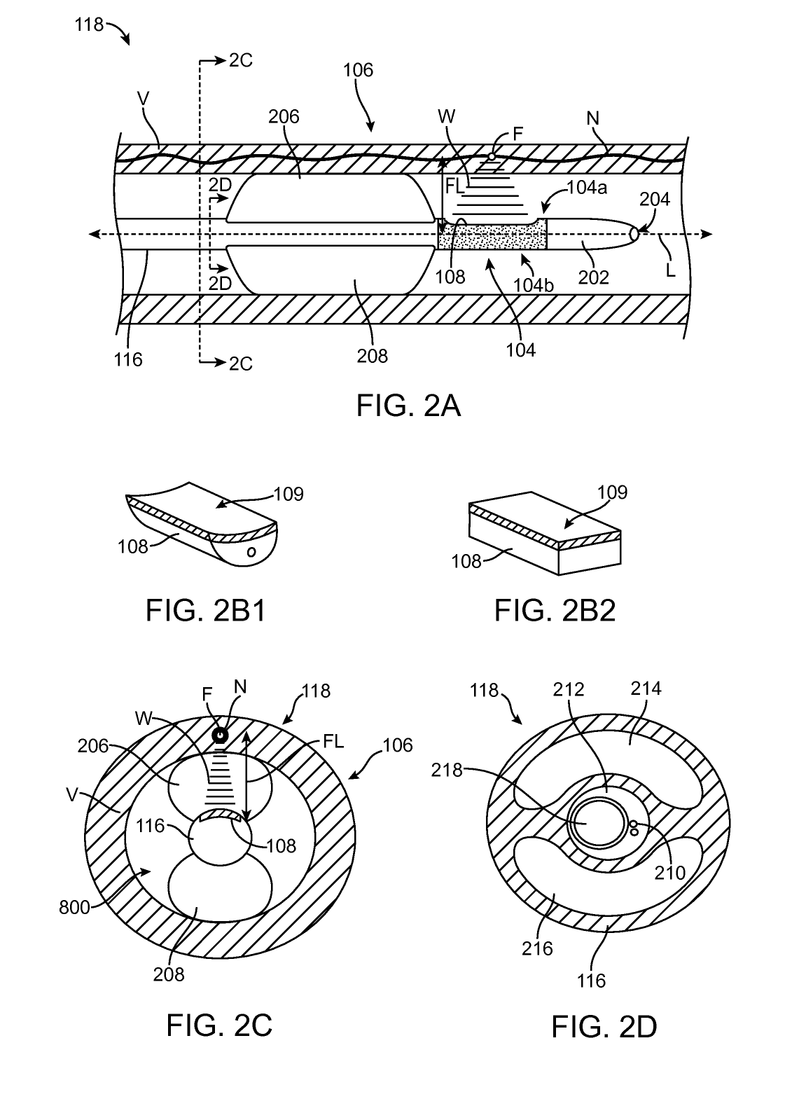

FIG. 1 is a partially schematic illustration of a neuromodulation system 100 (“system 100”) configured in accordance with an embodiment of the present technology. The system 100 includes an intravascular catheter 110 operably coupled to an energy source or energy generator 132 via a connector 130 (e.g., a cable). The catheter 110 can include an elongated shaft 116 having a proximal portion 114 and a distal portion 118. The catheter 110 also includes a handle assembly 112 at the proximal portion 114. The catheter 110 can further include a therapeutic assembly 104 carried by or affixed to the distal portion 118 of the elongated shaft 116 and a balloon assembly 106 at or adjacent to the therapeutic assembly 104 at the distal portion 118. The therapeutic assembly 104 and the balloon assembly 106 are described in greater detail below with reference to FIGS. 2A-5C. The elongated shaft 116 is configured to intravascularly locate the therapeutic assembly 104 at a treatment location within a renal blood vessel or within another suitable body lumen (e.g., within a ureter) of a human patient.

The energy source 132 can be configured to generate a selected form and/or magnitude of energy for delivery to the treatment site via a transducer 108 (FIG. 2A) of the therapeutic assembly 104. For example, the energy source 132 can include an energy generator (not shown) configured to generate ultrasound energy (e.g., focused ultrasound, high-intensity focused ultrasound (HIFU), etc.). The energy source 132 can be configured to control, monitor, supply, or otherwise support operation of the catheter 110. For example, a control mechanism, such as foot pedal 144, may be connected (e.g., pneumatically connected or electrically connected) to the energy source 132 to allow an operator to initiate, terminate and/or adjust various operational characteristics of the energy generator, such as ultrasound wave intensity and timing as well as balloon inflation volume and timing.

In some embodiments, the system 100 includes a remote control device (not shown) that can be configured to be sterilized to facilitate its use within a sterile field. The remote control device can be configured to control operation of the therapeutic assembly 104, the energy source 132, and/or other suitable components of the system 100. In other embodiments, the remote control device may be omitted and its functionality may be incorporated into the handle 112 or energy source 132.

As shown in FIG. 1, the energy source 132 can further include an indicator or display screen 136. The energy source can include other indicators, including one or more LEDs, a device configured to produce an audible indication, and/or other suitable communicative devices. In the embodiment shown in FIG. 1, the display 136 includes a user interface configured to receive information or instructions from a user and/or provide feedback to the user. For example, the energy source 132 can be configured to provide feedback to an operator before, during, and/or after a treatment procedure via the display 136. The feedback can be based on output from one or more sensors (not shown) associated with the therapeutic assembly 104 and/or balloon assembly 106 such as temperature sensor(s), impedance sensor(s), current sensor(s), voltage sensor(s), flow sensor(s), chemical sensor(s), ultrasound sensor(s), optical sensor(s), pressure sensor(s) and/or other sensing devices.

The system 100 can further include a controller 146 having, for example, memory (not shown) and processing circuitry (not shown). The memory and storage devices are computer-readable storage media that may be encoded with non-transitory, computer-executable instructions such as diagnostic algorithm(s) 133, control algorithm(s) 140, and/or evaluation/feedback algorithm(s) 138. The control algorithms 140 can be executed on a processor (not shown) of the system 100 to control the delivery of ultrasound energy to the therapeutic assembly 104, as well as to control inflation of one or more balloons of the balloon assembly 106. In some embodiments, selection of one or more parameters of an automated control algorithm 140 for a particular patient may be guided by diagnostic algorithms 133 that measure and evaluate one or more operating parameters prior to energy delivery. The diagnostic algorithms 133 provide patient-specific feedback to the clinician prior to activating the transducer 108 which can be used to select an appropriate control algorithm 140 and/or modify the control algorithm 140 to increase the likelihood of efficacious neuromodulation.

Although in the embodiment shown in FIG. 1 the controller 146 is incorporated into the energy source 132, in other embodiments the controller 146 may be an entity distinct from the energy source 132. For example, additionally or alternatively, the controller 146 can be personal computer(s), server computer(s), handheld or laptop device(s), multiprocessor system(s), microprocessor-based system(s), programmable consumer electronic(s), digital camera(s), network PC(s), minicomputer(s), mainframe computer(s), and/or any suitable computing environment.

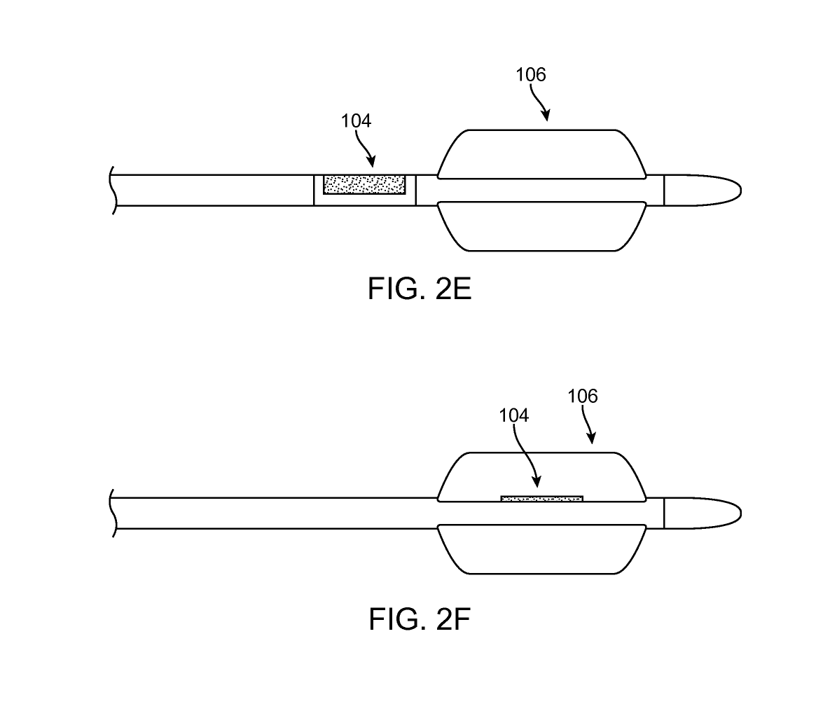

In some embodiments, the energy source 132 may include a pump 150 or other suitable pressure source (e.g., a syringe) operably coupled to an irrigation port 302 at the distal portion 118 of the catheter 110 (discussed in greater detail below with reference to FIG. 3A). In other embodiments, the pump 150 can be a standalone device separate from the energy source 132. Positive pressure generated by the pump 150 can be used, for example, to push a protective agent (e.g., saline) through the irrigation port 302 (FIG. 3A) to the treatment site. In yet other embodiments, the catheter 110 can include an adapter (not shown) (e.g., a luer lock) configured to be operably coupled to a syringe (not shown) and the syringe can be used to apply pressure to the shaft 116.

FIG. 2A shows an embodiment of the distal portion 118 of the catheter 110 deployed (e.g., inflated, expanded, etc.) within a blood vessel V. As shown, the distal portion 118 of the catheter 110 can include the therapeutic assembly 104 and the balloon assembly 106 carried by or affixed to the elongated shaft 116. The therapeutic assembly 104, for example, can be configured to emit ultrasound waves W and the balloon assembly 106 can be configured to position the distal portion 118 within the blood vessel V so that the emitted ultrasound waves W are focused on target depths where neural fibers N are likely to be located within the wall of the blood vessel V to induce thermal neuromodulation. In several embodiments, the distal portion 118 can include more than one therapeutic assembly 104 and/or more than one balloon assembly 106.

In the illustrated embodiment, the balloon assembly 106 is positioned at a location along the elongated shaft 116 proximal to the therapeutic assembly 104. Additionally or alternatively, the balloon assembly 106 can be positioned at a location along the elongated shaft 116 distal to the therapeutic assembly 104 (see, e.g., FIG. 2E) and/or positioned over at least a portion of the therapeutic assembly 104 (see, e.g., FIG. 2F). The distal portion 118 may terminate distally with, for example, an atraumatic, flexible tip 202 having an opening 204 at its distal end and in fluid connection with a central lumen 212 of the elongated shaft 116 (see FIG. 2C). The central lumen 212 can be configured to receive a guidewire slidably positioned therethrough. In some embodiments, the distal portion 118 may also be configured to engage another element of the system 100 or catheter 110.

FIG. 2C is a schematic cross-sectional end view of the distal portion 118 taken along the line 2C-2C in FIG. 2A. The cross-sectional view is taken along a longitudinal plane which can be defined as a plane perpendicular to a longitudinal axis L. Referring to FIGS. 2A and 2C together, the balloon assembly 106 can include two or more compliant balloons. In the illustrated embodiment, for example, the balloon assembly 106 includes a first balloon 206 and a second balloon 208 circumferentially offset from the first balloon 206 about the elongated shaft 116. As best seen in FIG. 2A, in some embodiments the first balloon 206 can be adjacent a first side 104a of the therapeutic assembly 104 and the second balloon 208 can be adjacent a second side 104b of the therapeutic assembly 104 that is opposite the first side 104a. Additionally, the first balloon 206 and the second balloon 208 may be positioned at generally the same location along the longitudinal axis L of the elongated shaft 116. In other embodiments, however, the first balloon 206 and the second balloon 208 may be positioned at different locations along the longitudinal axis L.

The one or more balloons of the balloon assembly 106 can individually extend less than 360 degrees around the shaft 116 (e.g., between about 90 degrees to about 180 degrees). The first and second balloons 206, 208 can be sized and shaped to allow fluid flow within the blood vessel V when in the deployed configuration. For example, the first and/or second balloons 206, 208 may only partially occlude the blood vessel V, leaving gaps 800 between the distal portion 118 and the vessel wall through which blood or other fluids may flow.

The shaft 116 can include inflation openings (not shown) for each balloon of the balloon assembly 106. The balloons can be laser-bonded or adhered by other suitable methods to an outer surface of the shaft 116 at radially spaced apart locations about the shaft 116. As best seen in the cross-sectional end view of the shaft 116 shown in FIG. 2D, individual inflation openings can connect to inflation lumens 214, 216 that extend proximally along the shaft 116 from the first and second balloon 206, 208 inflation openings, respectively, to the handle 112 (FIG. 1). The inflation lumens 214, 216 can be positioned within the shaft 116 at least proximate to opposite sides of the central lumen 212. In other embodiments, however, the inflation lumens 214, 216 may have other suitable shapes, sizes, and/or arrangements.

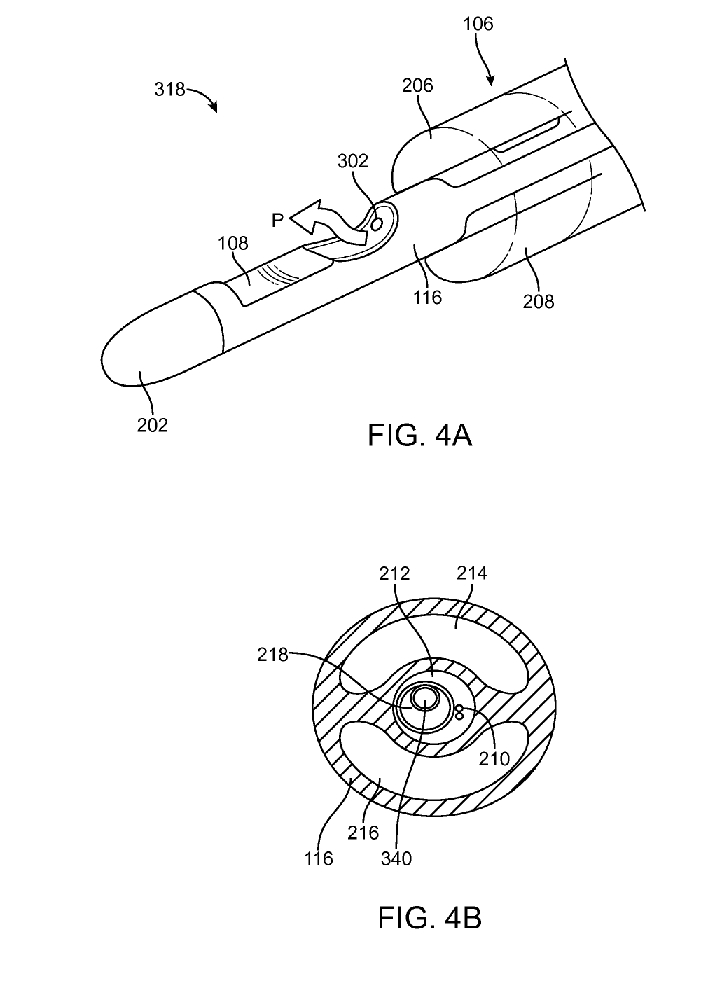

Referring again to FIGS. 2A-2D together, the therapeutic assembly 104 can include one or more ultrasound transducers 108 operably coupled to the energy source 132 via a conductor or wire 210 extending through a lumen 212 of the shaft 116. The transducer 108 is configured to deliver ultrasound energy W to neural fibers N located at least proximate to the wall of the blood vessel V at the treatment site. The transducer 108 may focus, collimate, or otherwise shape, direct, or redirect the emitted ultrasonic energy W in at least one direction, and may, when focusing, direct the ultrasonic energy W to a single focal point F or to a plurality of foci F. In some embodiments, the ultrasound transducer 108 is configured to transmit the ultrasound waves W over less than a full 360 degrees about the blood vessel V. Different foci F can be achieved by mechanical means such as lenses and/or reflectors (not shown), shaping and selection of the transducer material(s), and/or by electronic focusing such as frequency-changing methods wherein the focus location is a function of driving frequency. In a particular embodiment shown in the enlarged view of FIG. 2B1, the ultrasound transducer 108 is curved so that at least a surface 109 of the transducer 108 facing away from the elongated shaft 116 is concave to direct the ultrasonic energy to a focal point F. In other embodiments the transducer 108 may be any suitable shape or configuration (e.g., generally flat, cylindrical (FIG. 5A) convex, monotonically curvilinear, etc.).

At least a portion of the transducer can include a piezoelectric material, such as a piezoceramic, lead zirconate titanate (PZT), a piezocomposite, or a piezopolymer, and additionally or alternatively can include an electrostrictive material, a magnetostrictive material, a ferroelectric material, an electrostatic element, a micromechanical element, a micro-electro-mechanical element, a thermoplastic fluoropolymer (PVDF), or a combination thereof. In some embodiments, the transducer 108 can have a thickness between about 0.005 mm to about 0.020 mm, and a radius of about 1 mm to about 7 mm. In other embodiments, the radius may be about 3 mm to about 5 mm. In still further embodiments, the transducer 108 may have different dimensions and/or different features. For example, the transducer can be generally flat (FIG. 2B2) and have a thickness between about 0.005 inches and about 0.018 inches (e.g., 0.006 inches operating at a harmonic frequency of approximately 14.5 MHz, or 0.012 inches operating at a harmonic frequency of 7.5 MHz, etc.). In some embodiments, the transducer can be generally cylindrical having an outer diameter between about 0.050 inches and about 0.100 inches (e.g., 0.065 inches) and a wall thickness between about 0.010 inches and about 0.020 inches (e.g., 0.012 inches operating at a harmonic frequency of 7.5 MHz).

By focusing the ultrasound energy at one or more specific foci F within the tissue, the thermal effect of the focused ultrasound energy can be confined to a predetermined location or area, and such location or area can be remote from the ultrasound transducer and/or the wall of the blood vessel V. For example, the therapeutic assembly 104 can be configured to remain out of contact with the vessel wall during transmission of the ultrasound waves W to the neural fibers N, as shown in FIGS. 2A and 2C. Although the focal length FL (i.e., the radial distance between the transducer 108 and the focal point F) is generally constant and can be designed and/or accounted for, when the transducer 108 is radially centered within the vessel, the location of the transducer 108 relative to the wall of the blood vessel V and/or target nerves N is hard to predict because of varying anatomical and physiological conditions between patients. For example, referring to the schematic illustrations in FIGS. 3A-3B for explanatory purposes only, if a vessel V has a 2 mm inner radius IR and nerves N located 2 mm from the vessel wall, a transducer T having a focal length FL of 6 mm positioned at the radial center of the vessel V will have a focal point F that is 2 mm beyond the targeted nerve. Likewise, as shown in FIG. 3B, if the same transducer T is similarly positioned within a vessel V having a 6 mm inner radius IR and nerves N located 2 mm from the vessel wall, the focal point F would be 2 mm short of the targeted nerve N. In sum, if the transducer is incorrectly positioned: (1) the focal point F will not coincide with the target nerves N and thus the target nerves N may not receive sufficient ultrasound energy to affect therapeutic treatment, and (2) surrounding, non-target tissue (e.g., the vessel wall or beyond the vessel wall) may be inadvertently heated to damaging temperatures.

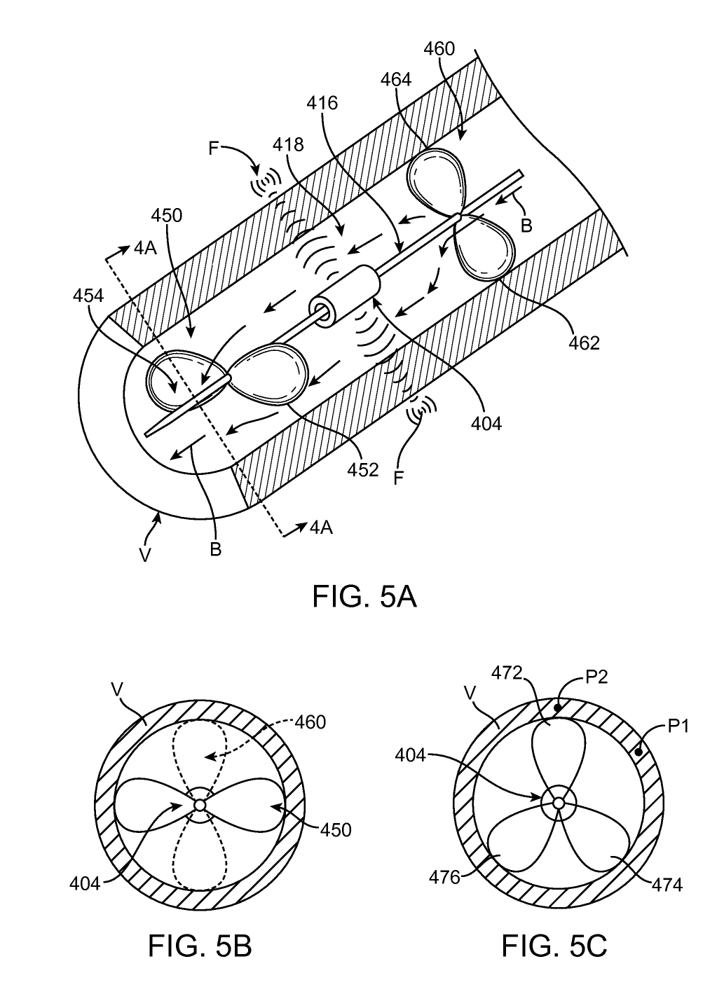

To address this need, the circumferentially offset, dual-balloon assembly of the present technology allows a clinician to precisely position the therapeutic assembly 104 within the blood vessel V based on anatomical parameters specific to the patient or a group of patients, such as vessel diameter, vessel wall thickness, focal length, location of the nerves with respect to the vessel wall, etc. Such parameters can be measured from pre-operative fluoroscopic images or, once the distal portion 118 is positioned at a target site, may be assessed with image guidance techniques (e.g., intravascular ultrasound (IVUS), computed tomography (CT), fluoroscopy, x-ray, optical coherence tomography (OCT), etc.). Accurate anatomical placement of the focal point F confines the therapeutic energy to a defined target area so that tissue in the target area is heated to a sufficiently high temperature to cause a desired thermal effect (e.g., tissue damage, ablation, coagulation, denaturation, destruction, necrosis, etc.) while tissue surrounding the target area is not heated to damaging temperatures and therefore is preserved. Devices and methods for control of the inflated volume and timing of inflation of the balloon assembly 106 is discussed in greater detail below with reference to FIGS. 6-11.

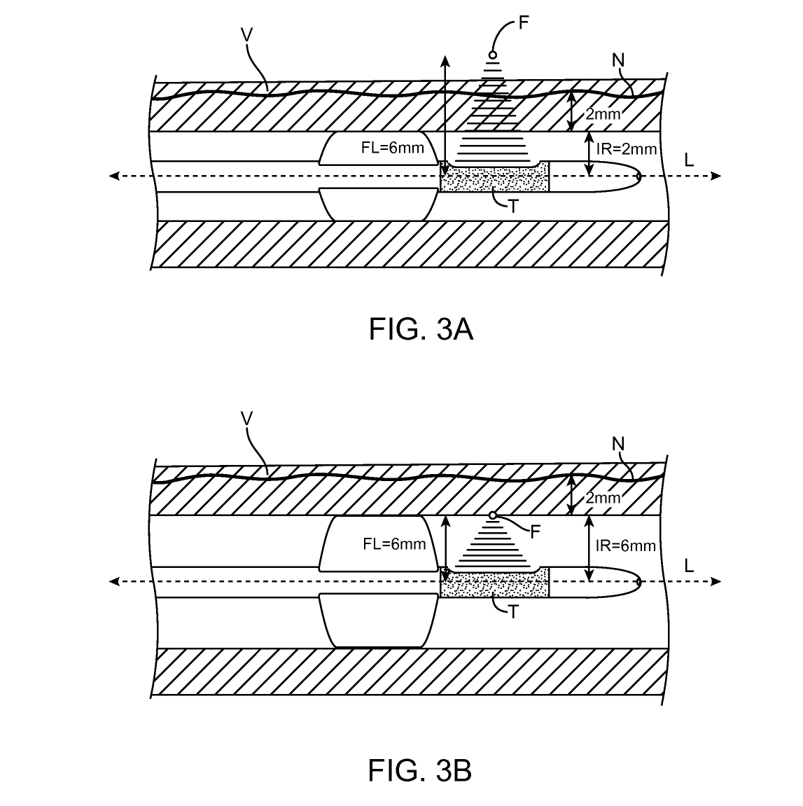

FIG. 4A illustrates another embodiment of a distal portion 318 of a catheter having an irrigation port 302 configured in accordance with the present technology. The irrigation port 302 can be configured to emit one or more protective agents P (e.g., saline) before, during, and/or after energy delivery to cool the transducer 108 and surrounding tissue. The irrigation port 302 may be located along the shaft 116 distal or proximal to the balloon assembly 106. As shown in the cross-sectional end view of the shaft 116 FIG. 4B, the irrigation port 302 can be in fluid connection with an irrigation lumen 340 that extends proximally along the shaft 116 from the irrigation port 302 to the handle 112 and/or energy source 132. In some embodiments, the catheter can include multiple irrigation ports, all in fluid communication with the irrigation lumen. In particular embodiments, the irrigation lumen 340 can be coupled to a pump 150 (see FIG. 1) or syringe (not shown) to facilitate conveyance of the protective agent P along the irrigation lumen 340 and irrigation of protective agent P through the irrigation port 302.

As discussed above, several embodiments of the catheter 110 can have multiple balloon assemblies. FIG. 5A, for example, is a side perspective view of a distal portion 418 of a therapeutic assembly having a first balloon assembly 450 and a second balloon assembly 460 configured in accordance with embodiments of the present technology. In the illustrated embodiment, the first balloon assembly 450 is positioned along the shaft 116 distal to the therapeutic assembly 404, and the second balloon assembly 460 is positioned along the shaft 116 proximal to the therapeutic assembly 404. In some embodiments, the first and second balloon assemblies 450, 460 may both be positioned distal and/or proximal to the therapeutic assembly 404. As shown in FIG. 5A, the first balloon assembly 450 can have a first balloon 452 circumferentially offset from a second balloon 454, and the second balloon assembly 460 can have a first balloon 462 circumferentially offset from a second balloon 464. The first balloon assembly 450 and the second balloon assembly 460 may each have the same number of balloons or they may have a different number of balloons.

As shown in the cross-sectional end view of FIG. 5B, the balloons of the first balloon assembly 450 can be circumferentially offset from the balloons of the second balloon assembly 460 such that when the balloons are expanded, fluids B (e.g., blood, infusate, etc.) may continue to move through the blood vessel V at the treatment site to provide a natural cooling source for the vessel. In the illustrated embodiments, the first balloon assembly 450 and the second balloon assembly 460 are circumferentially offset by about 90 degrees. In other embodiments, the first balloon assembly 450 and the second balloon assembly 460 can be circumferentially offset by more or less than 90 degrees such that expanding the first balloon 452, first balloon 462, second balloon 454 and/or second balloon 464 to different volumes selectively repositions the transducer 404 within the vessel. In one embodiment, the first balloon assembly 450 and the second balloon assembly 460 are not offset and are substantially aligned.

Additionally, as shown in FIG. 5C, in some embodiments each of the first and second balloon assemblies 450, 460 can have three balloons. For example, the first and/or second balloon assemblies 450, 460 can have a first balloon 472, a second balloon 474, and a third balloon 476. Any or all of the first, second and/or third balloons 472, 474, 476 can be expanded/inflated to the same or different volumes such that the transducer 408 can be selectively positioned closer to or farther from any point along the circumference of the vessel V. For example, in the illustrated embodiment, selectively expanding the first balloon 472 and the second balloon 474 to a lesser volume than the third balloon 476 moves the transducer 408 closer to a point P1 along the vessel wall V. Similarly, selectively expanding the second balloon 474 and the third balloon 476 to a lesser volume than the first balloon 472 moves the transducer 408 closer to a point P2 along the vessel wall V.

In other embodiments, the first and second balloon assemblies 450, 460 can individually have different numbers of balloons. For example, in one embodiment the first balloon assembly can have two balloons and the second balloon assembly can have three balloons. In these and other embodiments, the first and/or second balloon assemblies 450, 460 can have any suitable number of balloons (e.g., one balloon, four balloons, five balloons, etc.).

### III. SELECTED DELIVERY METHODS

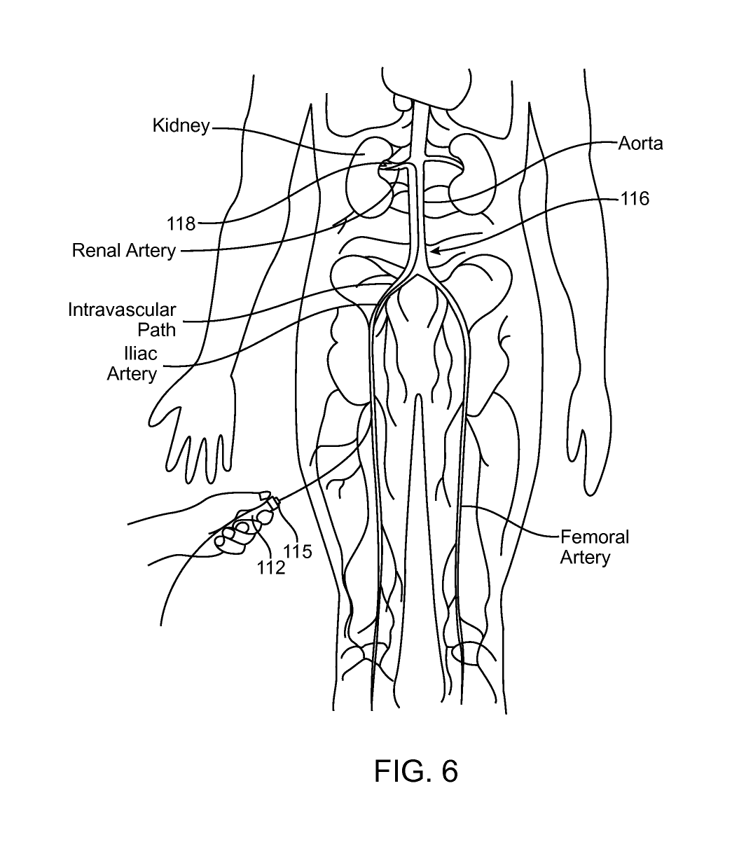

FIG. 6 (with additional reference to FIG. 1) illustrates modulating renal nerves with an embodiment of the system 100. Intravascular delivery of the distal portion 118 of the catheter 110 can include percutaneously inserting a guide wire 115 within the vasculature at an access site (e.g., femoral, brachial, radial, or axillary artery) and moving the shaft and the distal portion 118 (in the delivery state) along the guidewire 115 until at least a portion of the therapeutic assembly 104 reaches the treatment location (as shown in FIG. 7). In some embodiments, the central lumen 212 (FIG. 2D) extending through the shaft 116 and the distal portion 118 can be configured to receive the guide wire 115 in an over-the-wire configuration. In other embodiments, the shaft 116 and the distal portion 118 can be configured in a rapid exchange configuration. The guide wire 115 may comprise any suitable medical guide wire sized to slideably fit within the lumen 212. In one particular embodiment, for example, the guide wire 115 may have a diameter of 0.356 mm (0.014 inch). As illustrated, a section of the proximal portion of the shaft 116 can be externally positioned and manipulated by the operator to advance the shaft 116 through the sometimes tortuous intravascular path and remotely manipulate the distal portion 118 of the shaft 116. In other embodiments, the distal portion 118 may be delivered to the treatment site within a guide sheath (not shown) with or without using the guide wire 115. When the therapeutic assembly 104 (FIG. 1) is at the treatment site, the guide sheath may be at least partially withdrawn or retracted and the therapeutic assembly 104 can be transformed into the deployed arrangement. In still other embodiments, the shaft 116 may be steerable itself such that the therapeutic assembly 104 at the distal portion 118 may be delivered to the treatment site without the aid of the guide wire 115 and/or guide sheath.

Image guidance, e.g., CT, fluoroscopy, IVUS, OCT, intracardiac echocardiography (ICE), or another suitable guidance modality, or combinations thereof, may be used to aid the clinician's positioning and manipulation of the distal portion 118 and/or the therapeutic assembly 104. For example, a fluoroscopy system (e.g., including a flat-panel detector, x-ray, or c-arm) can be rotated to accurately visualize and identify the target treatment site. In other embodiments, the treatment site can be located using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the catheter 110. Further, in some embodiments, image guidance components (e.g., IVUS, OCT) may be integrated with the catheter 110 and/or run in parallel with the catheter 110 to provide image guidance during positioning of the distal portion 118, balloon assembly 106, and/or therapeutic assembly 104. For example, image guidance components (e.g., IVUS or OCT) can be coupled to a distal portion of the catheter 110 to provide three-dimensional images of the vasculature proximate the target site to facilitate positioning or deploying the distal portion 118 within the target renal blood vessel.

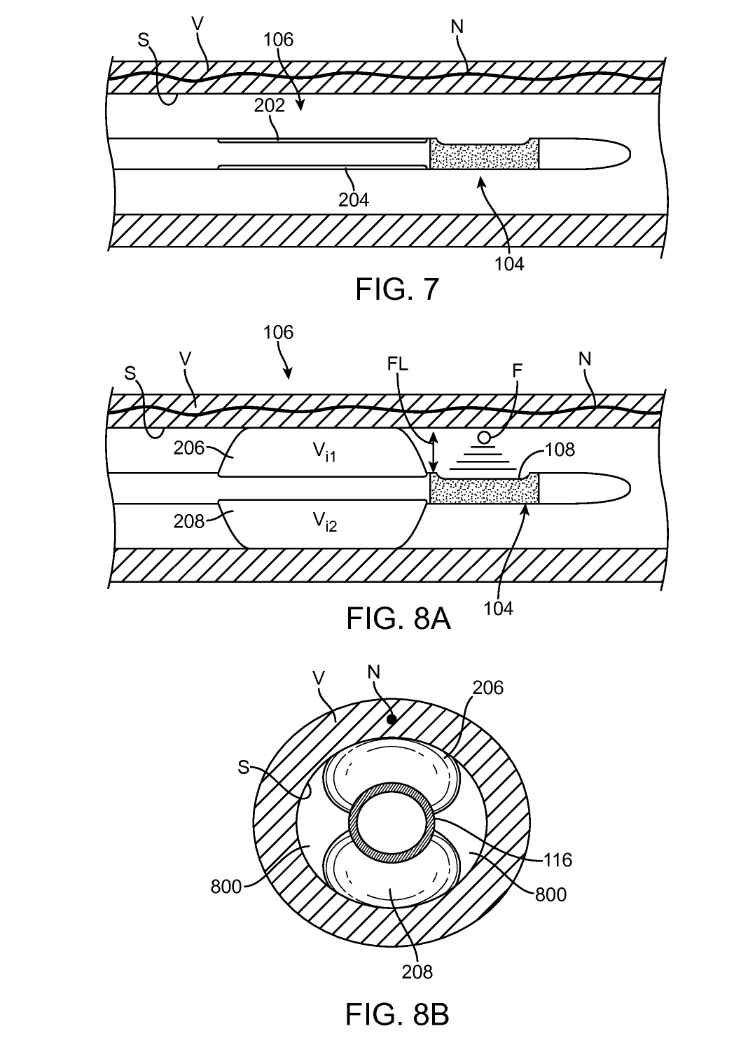

Referring to FIGS. 8A-8B together, once the distal portion 118 is positioned at the treatment location, the individual balloons of the balloon assembly 106 can be at least partially inflated or expanded to transform the balloon assembly 106 from a low-profile delivery configuration to a deployed or expanded configuration. The first balloon 206 and the second balloon 208 may be inflated simultaneously or sequentially to a first initial volume Vᵢ₁ and a second initial volume Vᵢ₂, respectively. The first and second balloons 206, 208 may be inflated to the same or different initial volumes depending on the desired positioning within the vessel. For example, depending on the inner diameter of the vessel at the treatment site, the focal length FL may be more or less than the radial distance between the transducer 108 and the target nerves N. Accordingly, inflating the balloons to approximately the same initial volumes Vᵢ₁, Vᵢ₂ such that the transducer is radially centered within the vessel V could still result in the focal point F not coinciding with the target nerve location.

FIG. 8A shows one scenario where inflating the balloons to approximately the same initial volumes Vᵢ₁, Vᵢ₂ results in the focal point F being short of the target nerve location. To address this difference, as shown in FIG. 8B, the volume of the balloon located on the side of the shaft 116 adjacent to the focal point F (e.g., first balloon 206) may be decreased (e.g., automatically or manually) to a new volume Vₙ₁ while the volume of the balloon located on the side of the shaft 116 opposite the focal point F (e.g., second balloon 208) may be increased to a new volume Vₙ₂ to bring the therapeutic assembly 104 closer to the portion of the vessel wall proximate to the target nerves N and the focal point F within the target region. As a result, once the clinician activates the transducer 108, the focal point F of the emitted ultrasound energy will be generally concurrent with the target nerves N.

In some embodiments (not shown), inflating the balloons to approximately the same initial volumes Vᵢ₁, Vᵢ₂ can result in the focal point F being too deep and thus beyond the target nerve location. In this case, the volume of the balloon located on the side of the shaft 116 adjacent to the focal point F may be increased (e.g., automatically or manually) to a new volume (not shown) while the volume of the balloon located on the side of the shaft 116 opposite the focal point F may be decreased to a new volume (not shown) to reposition the therapeutic assembly 104 farther from the portion of the vessel wall proximate to the target nerves N and the focal point F within the target region.

In some procedures it may be necessary to adjust the positioning of the transducer 108 multiple times. For example, the first and/or second balloon volumes V₁, V₂ can be adjusted (e.g., increased and decreased) any number of times to move the therapeutic assembly 104 and/or transducer 108 closer to or farther from the wall of the blood vessel V proximate the target nerves N in order to create several ablations at different depths. Additionally, the first and/or second balloon volumes V₁, V₂ can be decreased so that the clinician may reposition the distal portion 118 to target previously untargeted nerves, or to adjust the approach for the same targeted nerve. The clinician may reposition the distal portion 118 by rotating the therapeutic assembly 104 about the longitudinal axis of the shaft, longitudinally moving the distal portion 118 distally or proximally relative to the vessel wall, and/or by any change in position of the therapeutic assembly 104 with respect to the vessel wall. After repositioning, the clinician may re-inflate the first and/or second balloons 206, 208 so that the focal length FL is generally equivalent to the radial distance between for the therapeutic assembly 104 to the targeted nerves N and re-activate the therapeutic assembly 104 to modulate the nerves N.

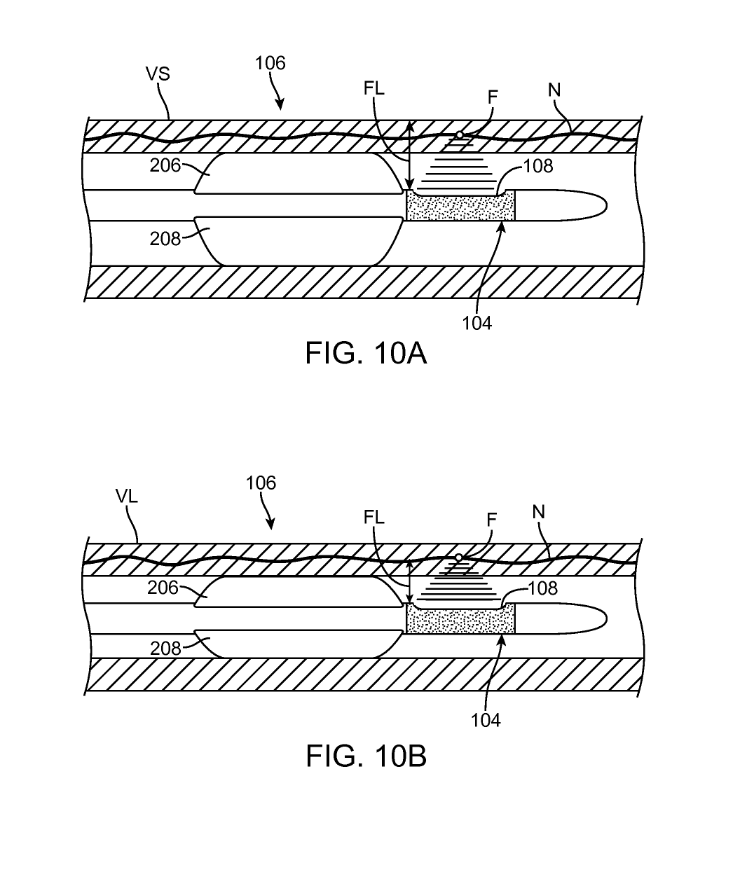

In some embodiments the balloon assembly 106 may be used within the same procedure to position the therapeutic assembly 104 in different vessels having different diameters and/or different portions of the same vessel having different diameters (e.g., branch arteries). FIG. 10A, for example, illustrates the distal portion 118 positioned within a relatively narrow vessel or portion of a vessel with the volume of the first and second balloons tailored to accommodate the smaller diameter of the vessel VS and its effect on the radial distance between the therapeutic assembly 104 and the wall of the vessel VS. After modulating target nerves N at a treatment site within the narrower vessel or portion of a vessel VS, the clinician may deflate the first and/or second balloons 206, 208 and reposition the distal portion 118 in a vessel or a portion of a vessel having a larger diameter. Once again, the first and/or second balloons volumes can be dynamically increased and/or decreased to coordinate the focal length FL with the radial distance between the transducer 108 and the target nerves N before activating the therapeutic assembly to modulate nerves at a treatment site in the vessel or portion of a vessel having a relatively larger diameter VL (FIG. 10B).

Control of the first or second balloon 206, 208 (e.g., control over inflation/expansion volume, inflation/expansion timing and/or deflation/collapse timing) can be manual or automatic. For example, the volume and inflation/deflation timing can be based on a pre-set schedule or algorithm that automatically expands one, a few, or all balloons to a specific, desired expanded volume and/or outer diameter based on the focal length FL of the transducer and the location of the target nerves relative to the vessel. The clinician may be prompted by the display 136 (FIG. 1) to input a value (e.g., via the display or interactive user interface) corresponding to the size of the vessel or another suitable parameter or group of parameters (e.g., pressure, flow rates, temperature, etc.). The vessel diameter, vessel wall thickness, and/or location or depth of the target nerves N can be measured from pre-operative fluoroscopic images and/or by the already discussed intravascular imaging techniques.

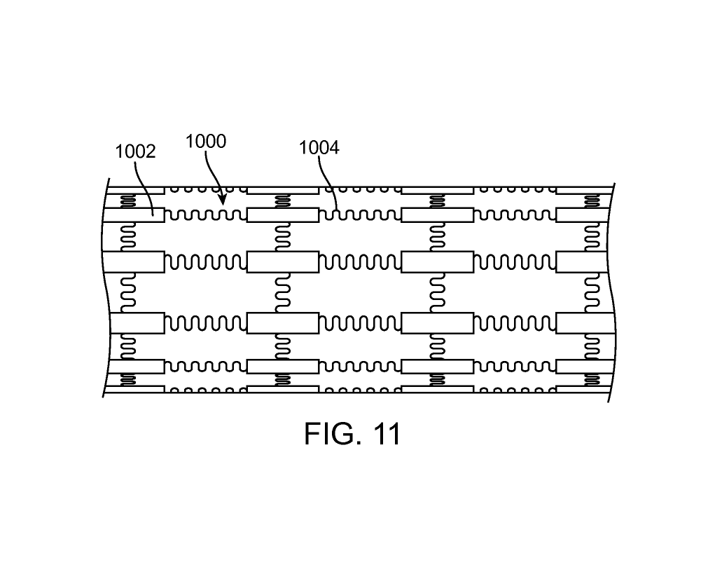

In some embodiments, one or more pressure sensors (e.g., micro-flow controllers, serially synchronized pressure sensors, pressure tubes, etc.) may be provided so that the balloon automatically expands to an appropriate expanded volume without the need to input a vessel or lumen size prior to inflation. FIG. 11, for example, is an enlarged view of a portion of a balloon 1000 configured in accordance with an embodiment of the technology. The balloon 1000 may be fully or partially fitted with one or more pressure sensors 1002 that are interconnected by one or more stretchy, serpentine-shaped wires 1004 configured to buckle and/or adapt as the balloon 1000 expands/inflates and/or collapses. The pressure sensors 1002 can be electrically coupled to the handle 112 and/or the console 132 by one or more wires (not shown) extending through the shaft 116. The console 132 may include one or more customizable algorithms that detect an increase and/or decrease in pressure exerted on the pressure sensors 1002 by the interconnecting wires 1004 (e.g., as the balloon 1000 expands/inflates and/or collapses) to control inflation of the balloon 1000. In some embodiments, the pressure sensors 1002 could be used to measure the vessel diameter so that a user would not need to input vessel size. For example, the measured vessel diameter may be used for the purpose of controlling expansion/inflation of the balloons. In one embodiment, the balloons could be expanded/inflated until registering a pre-determined threshold pressure or volume that correlates to a particular vessel diameter.

### IV. PERTINENT ANATOMY AND PHYSIOLOGY

The following discussion provides further details regarding pertinent patient anatomy and physiology. This section is intended to supplement and expand upon the previous discussion regarding the relevant anatomy and physiology, and to provide additional context regarding the disclosed technology and the therapeutic benefits associated with renal neuromodulation. For example, as mentioned previously, several properties of the renal vasculature may inform the design of catheters and associated methods for achieving renal neuromodulation, and impose specific design requirements for such devices. Specific design requirements may include accessing the renal artery, ureter, or renal pelvic anatomy, facilitating stable contact between a therapeutic element of a catheter and a luminal surface or wall, and/or effectively modulating the renal nerves using the therapeutic element.

A. The Sympathetic Nervous System

The SNS is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS extend through tissues in almost every organ system, providing at least some regulatory function to characteristics as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

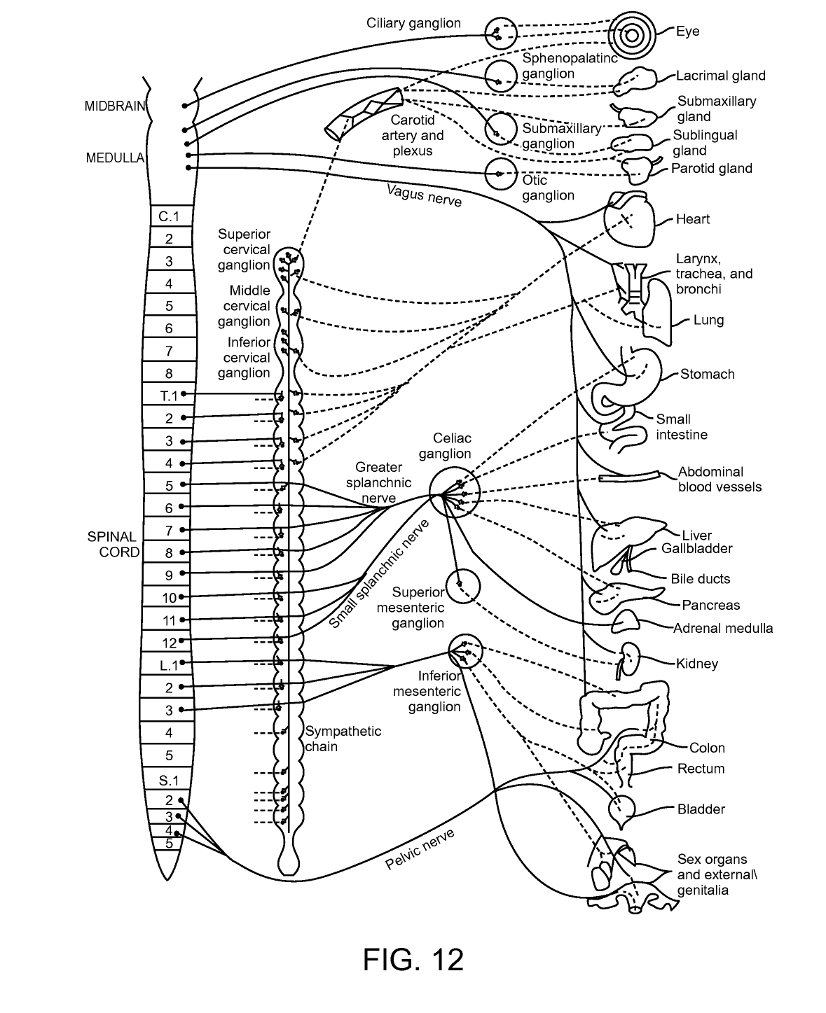

As shown in FIG. 12, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which send sympathetic nerve fibers to the head and thorax organs, and the celiac and mesenteric ganglia (which send sympathetic fibers to the gut).

2. Nerves of the Kidneys

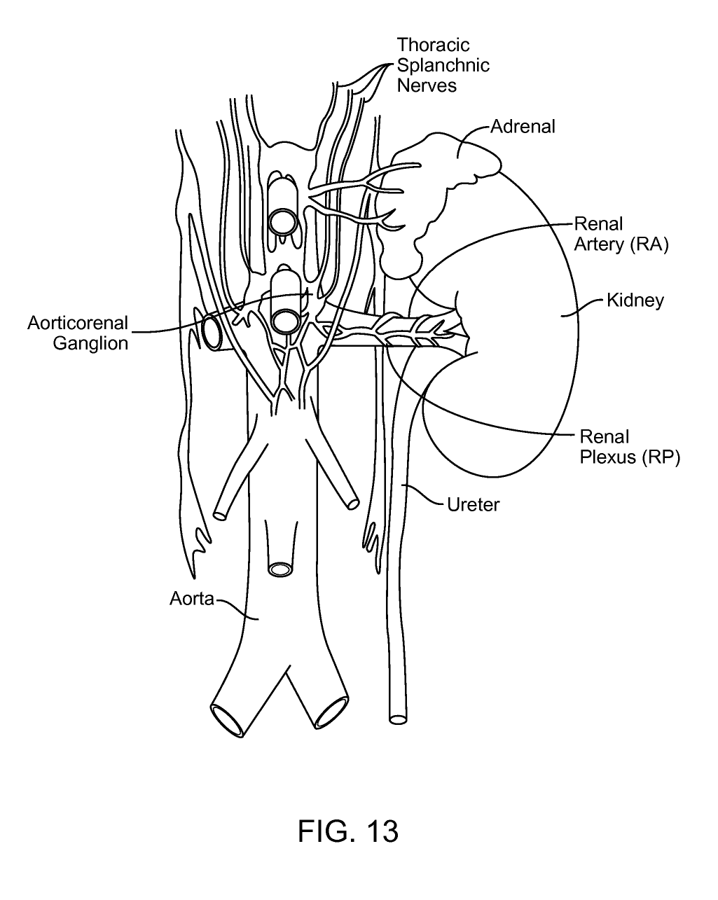

As shown in FIG. 13, the kidney neural system includes the renal plexus, which is intimately associated with the renal artery. The renal plexus is an autonomic plexus that surrounds the renal artery and is embedded within the adventitia of the renal artery. The renal plexus extends along the renal artery until it arrives at the substance of the kidney. Fibers contributing to the renal plexus arise from the celiac ganglion, the superior mesenteric ganglion, the aorticorenal ganglion and the aortic plexus. The renal plexus, also referred to as the renal nerve, is predominantly comprised of sympathetic components. There is no (or at least very minimal) parasympathetic neural activity of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

### i. Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

### ii. Renal Sensory Afferent Nerve Activity

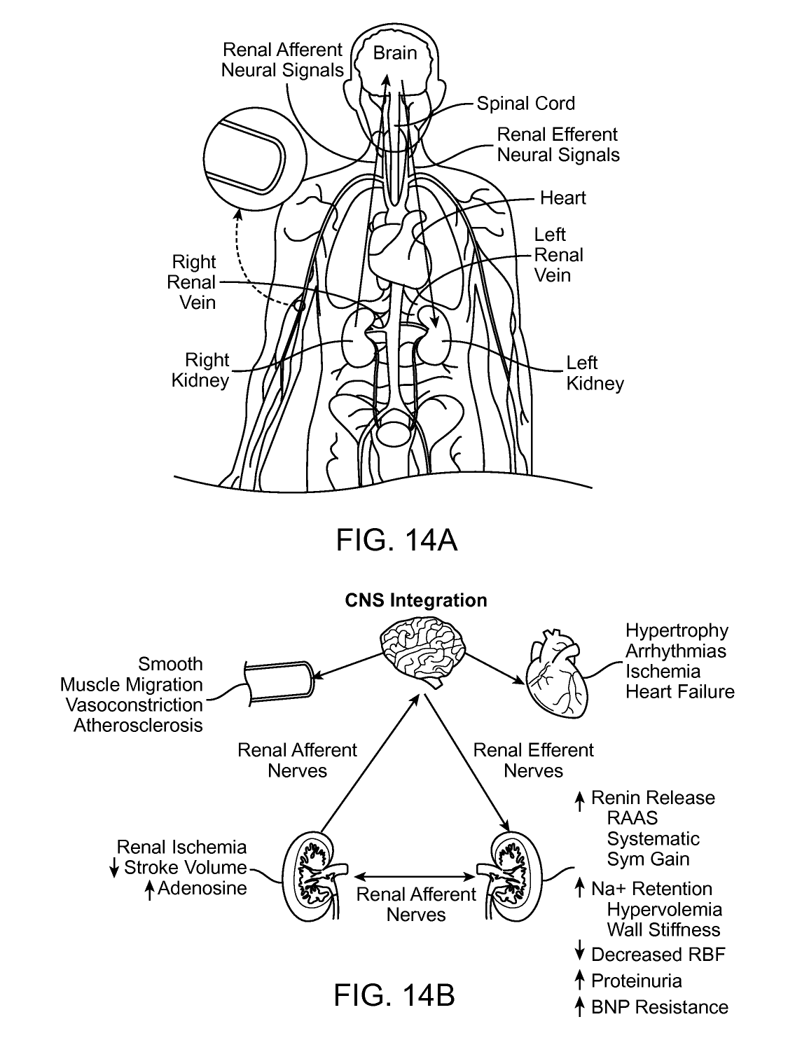

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine may trigger activation of afferent neural communication. As shown in FIGS. 14A and 14B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, fluid volume retention, and vasoconstriction. Central sympathetic over activity also impacts other organs and bodily structures having sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, sodium retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal neuromodulation, a desirable reduction of central sympathetic outflow to various other organs such as the heart and the vasculature is anticipated.

B. Additional Clinical Benefits of Renal Neuromodulation

As provided above, renal neuromodulation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal neuromodulation might also be useful in treating other conditions associated with systemic sympathetic hyperactivity. Accordingly, renal neuromodulation may also benefit other organs and bodily structures having sympathetic nerves, including those identified in FIG. 12.

C. Achieving Intravascular Access to the Renal Artery

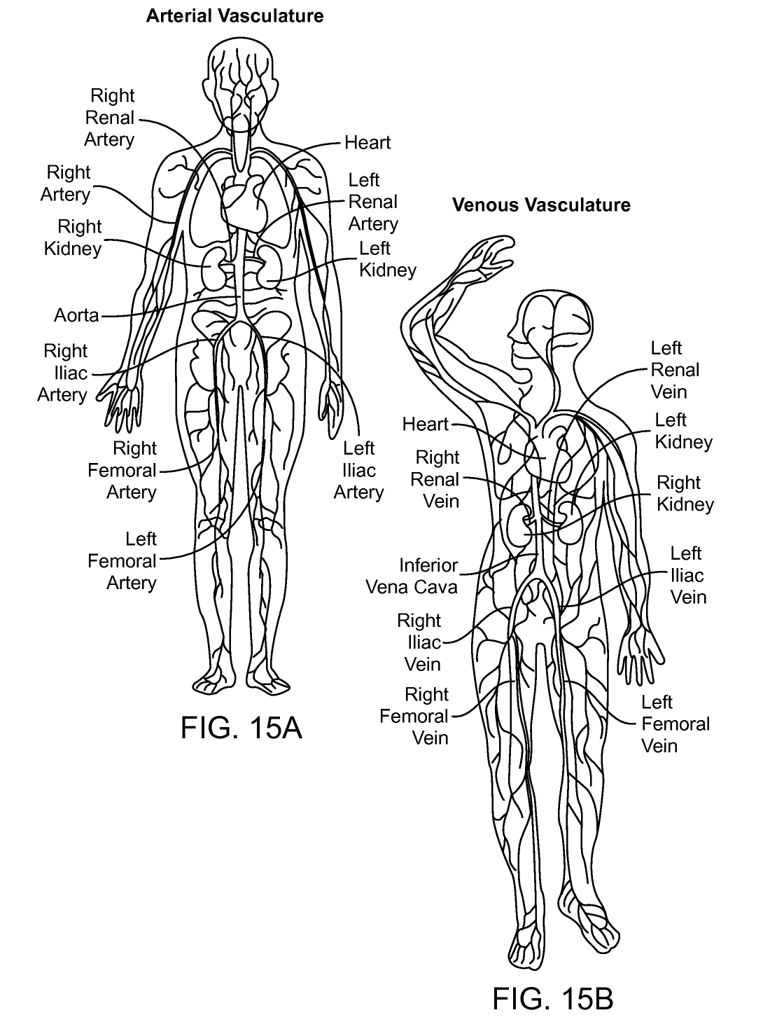

In accordance with the present technology, neuromodulation of a left and/or right renal plexus RP, which is intimately associated with a left and/or right renal artery, may be achieved through intravascular access. As FIG. 15A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 15B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, consistent positioning and appropriate contact force applied by the energy delivery element to the vessel wall can be important for predictability. However, navigation typically is impeded by the tight space within a renal artery, as well as tortuosity of the artery. Furthermore, establishing consistent contact can be complicated by patient movement, respiration, and/or the cardiac cycle. These factors, for example, may cause significant movement of the renal artery relative to the aorta, and the cardiac cycle may transiently distend the renal artery (i.e., cause the wall of the artery to pulse).

After accessing a renal artery and facilitating stable contact between neuromodulatory apparatus and a luminal surface of the artery, nerves in and around the adventitia of the artery can be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that renal nerves may be multiple millimeters distant from the luminal surface of the artery. Sufficient energy can be delivered to the renal nerves to modulate the renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Accordingly, the complex fluid mechanics and thermodynamic conditions present in the renal artery during treatment, particularly those that may impact heat transfer dynamics at the treatment site, may be important in applying energy from within the renal artery.

The neuromodulatory apparatus can be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

Blood flow through a renal artery may be temporarily occluded for a short time with minimal or no complications. However, occlusion for a significant amount of time can be avoided in some cases to reduce the likelihood of injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the take-off angle of a renal artery relative to the aorta. These properties will be discussed in greater detail with respect to the renal arteries. However, depending on the apparatus, systems, and methods utilized to achieve renal neuromodulation, such properties of the renal arteries also may guide and/or constrain design characteristics.

As noted above, an apparatus positioned within a renal artery can conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the renal plexus is embedded within the adventitia of the renal artery, the composite Intima-Media Thickness, IMT, (i.e., the radial outward distance from the artery's luminal surface to the adventitia containing neural structures) also is notable and generally is in a range of about 0.5-2.5 mm, with an average of about 1.5 mm. Although a certain depth of treatment can be important to reach the neural fibers, the treatment can be prevented from becoming too deep (e.g., >5 mm from inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, which located at the distal end of the renal artery, may move as much as 4 inches cranially with respiratory excursion. This may impart significant motion to the renal artery connecting the aorta and the kidney, thereby requiring from the neuromodulatory apparatus a unique balance of stiffness and flexibility to maintain contact between the thermal treatment element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### V. EXAMPLES

1. An ultrasound apparatus for thermally-induced neuromodulation, the apparatus comprising:
an elongated tubular shaft having a proximal portion and a distal portion;
a therapeutic assembly at the distal portion of the elongated shaft and adapted to be located within a blood vessel of a human patient at a treatment site, the therapeutic assembly comprising an ultrasound transducer configured to transmit ultrasound waves; and
a balloon assembly at the distal portion of the elongated shaft and adjacent to the therapeutic assembly, wherein the balloon assembly comprises a first balloon and a second balloon circumferentially offset from the first balloon, and wherein the first and second balloons are configured to vary between a delivery configuration and a deployed configuration,
wherein, when the first and second balloons are in the deployed configuration, the balloon assembly is configured to selectively position the ultrasound transducer at a desired location within the blood vessel such that the ultrasound waves from the ultrasound transducer are focused on neural fibers within the blood vessel wall to induce thermal neuromodulation.

2. The ultrasound apparatus of example 1 wherein the desired location comprises a selected radial distance between a surface of the transducer and the neural fibers.

3. The ultrasound apparatus of any one of examples 1 and 2 wherein:
the transducer comprises a first side and a second side opposite the first side; and
the first balloon is adjacent the first side of the transducer and the second balloon is adjacent the second side of the transducer.

4. The ultrasound apparatus of any one of examples 1 to 3 wherein, when in the deployed configuration, the first balloon has a first volume and the second balloon has a second volume different than the first volume.

5. The ultrasound apparatus of any one of examples 1 to 4 wherein, when in the deployed configuration, the first balloon and the second balloon have the same or approximately the same volume.

6. The ultrasound apparatus of any one of examples 1 to 5 wherein the first and second balloons are sized and shaped to allow fluid flow within the blood vessel when in the deployed configuration.

7. The ultrasound apparatus of any one of examples 1 to 6 wherein the balloon assembly is located proximal of the transducer assembly along the elongated shaft.

8. The ultrasound apparatus of any one of examples 1 to 7 wherein the balloon assembly is positioned over at least a portion of the transducer assembly along the elongated shaft.

9. The ultrasound apparatus of any one of examples 1 to 8 wherein the ultrasound transducer is curved.

10. The ultrasound apparatus of any one of examples 1 to 9 wherein the ultrasound transducer is configured to transmit the ultrasound waves over less than a full 360° about the blood vessel.

11. The ultrasound apparatus of any one of examples 1 to 10 wherein the therapeutic assembly is configured to remain out of contact with a wall of the blood vessel during transmission of the ultrasound waves to the target depth.

12. The ultrasound apparatus of any one of examples 1 to 11 wherein the elongated tubular shaft and the therapeutic assembly together define therethrough a guide wire lumen configured to slidably receive a medical guide wire, and wherein the therapeutic assembly is configured to be delivered via guide wire to the treatment site within the blood vessel.

13. The ultrasound apparatus of any one of examples 1 to 12 wherein the therapeutic assembly is adapted to be located at a treatment site within a renal artery of the patient, and wherein the ultrasound transducer is configured to deliver the ultrasound waves to renal nerves along the renal artery.

14. A catheter apparatus, comprising:
an elongated shaft having a proximal portion and a distal portion; and
an ultrasound transducer at the distal portion of the elongated shaft and configured for intravascular placement within a renal blood vessel of a human patient, wherein the ultrasound transducer is configured to transmit ultrasound waves to renal nerves along the renal blood vessel to achieve thermal renal neuromodulation via heating;
a first balloon assembly at the distal portion of the elongated shaft and between the ultrasound transducer and a distal end of the elongated shaft; and
a second balloon assembly at the distal portion of the elongated shaft and located proximally of the ultrasound transducer along the elongated shaft,
wherein the first balloon assembly and the second balloon assembly are configured to vary between a low-profile delivery state and an expanded deployed state,
wherein, when the first balloon assembly and the second balloon assembly are in the deployed state, the first balloon assembly and the second balloon assembly are sized and shaped to position the ultrasound transducer within the renal blood vessel at a desired focal length from the renal nerves and out of contact with an interior wall of the renal blood vessel, and
wherein, when the first balloon assembly and the second balloon assembly are in the deployed state, the first balloon assembly and the second balloon assembly are sized and shaped to allow blood flow through the renal blood vessel.

15. The catheter apparatus of example 15 wherein the distal portion is adapted to be located within a renal artery of the patient, and wherein the ultrasound transducer is configured to deliver the ultrasound waves to renal nerves along the renal artery.

16. The catheter apparatus of any one of examples 14 and 15 wherein:
the first balloon assembly further comprises a first plurality of balloons, wherein each of the first plurality of balloons is circumferentially offset from one another about the elongated shaft; and
the second balloon assembly further comprises a second plurality of balloons, wherein each of the second plurality of balloons is circumferentially offset from one another about the elongated shaft.

17. A method, comprising:
positioning a distal portion of a catheter at a treatment site within or otherwise proximate to a vessel having a neural fiber proximate a wall of the vessel, wherein the distal portion includes—
   a therapeutic assembly having an ultrasound transducer with a focal length, and
   a balloon assembly having a first balloon and a second balloon circumferentially offset from the first balloon;
expanding the first balloon to a first volume;
expanding the second balloon to a second volume such that the transducer and the neural fiber are separated by the focal length; and
activating the therapeutic assembly to modulate the neural fiber.

18. The method of example 17 wherein the first volume and the second volume are different.

19. The method of any one of examples 17 and 18 wherein expanding the second balloon further comprises bringing the transducer closer to a portion of the wall of the vessel proximate to the neural fiber.

20. The method of any one of examples 17 to 19 wherein activating the therapeutic assembly occurs at a first time, and wherein the method further comprises:

decreasing the first and/or second volumes to deflate the first and/or second balloons, respectively;
rotating the therapeutic assembly about a longitudinal axis of the shaft;
reinflating the first balloon and the second balloon such that the transducer and the neural fiber are separated by the focal length; and
activating the therapeutic assembly to modulate nerves of the patient at a second time.

21. The method of any one of examples 17 to 19 wherein:
the neural fiber is a first neural fiber, and
activating the therapeutic assembly occurs at a first time; and
wherein the method further comprises—
   decreasing the first and/or second volumes to deflate the first and/or second balloons, respectively;
   rotating the therapeutic assembly about a longitudinal axis of the shaft;
   reinflating the first balloon and the second balloon such that the transducer and a second neural fiber are separated by the focal length, wherein the second neural fiber is different than the first neural fiber; and
   activating the therapeutic assembly to modulate nerves of the patient at a second time.

22. A method, comprising:
positioning a distal portion of an intravascular catheter at a first treatment site within or otherwise proximate to a vessel of a human patient, wherein the first vessel at the treatment site has a first diameter, and wherein the distal portion includes—
   a therapeutic assembly configured to deliver ultrasound energy to neural fibers proximate a wall of the vessel, and
   a balloon assembly having a first balloon and a second balloon;
expanding the first balloon to a first balloon first volume and the second balloon to a second balloon first volume, wherein the first balloon first volume is the same or different than the second balloon first volume;
activating the therapeutic assembly to modulate nerves of the patient;
removing the therapeutic assembly from the first treatment site;
positioning the distal portion at a second treatment site having a second diameter different than the first diameter;
expanding the first balloon to a first balloon second volume, that is the same or different than the first balloon first volume;
expanding the second balloon to a second balloon second volume that is the same or different than the first balloon first volume; and
activating the therapeutic assembly to modulate nerves of the patient at a second time.

23. The method of example 22 wherein the first treatment site and the second treatment site comprise different portions of the vessel.

24. The method of example 22 wherein the vessel is a first vessel, and wherein the first treatment site is located at the first vessel, and the second treatment site is located at a second vessel different than the first vessel.

### VI. CONCLUSION

The above detailed descriptions of embodiments of the present technology are for purposes of illustration only and are not intended to be exhaustive or to limit the present technology to the precise form(s) disclosed above. Various equivalent modifications are possible within the scope of the present technology, as those skilled in the relevant art will recognize. For example, while steps may be presented in a given order, alternative embodiments may perform steps in a different order. The various embodiments described herein and elements thereof may also be combined to provide further embodiments. In some cases, well-known structures and functions have not been shown or described in detail to avoid unnecessarily obscuring the description of embodiments of the present technology.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Certain aspects of the present technology may take the form of computer-executable instructions, including routines executed by a controller or other data processor. In some embodiments, a controller or other data processor is specifically programmed, configured, and/or constructed to perform one or more of these computer-executable instructions. Furthermore, some aspects of the present technology may take the form of data (e.g., non-transitory data) stored or distributed on computer-readable media, including magnetic or optically readable and/or removable computer discs as well as media distributed electronically over networks. Accordingly, data structures and transmissions of data particular to aspects of the present technology are encompassed within the scope of the present technology. The present technology also encompasses methods of both programming computer-readable media to perform particular steps and executing the steps.

Additionally, instructions, data structures, and message structures of the controller 146 may be stored or transmitted via a data transmission medium, such as a signal on a communications link and may be encrypted. Various communications links may be used, such as the Internet, a local area network, a wide area network, a point-to-point dial-up connection, a cell phone network, Bluetooth, RFID, and other suitable communication channels. The system 100 may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An ultrasound apparatus for thermally-induced neuromodulation, the apparatus comprising:
an elongated tubular shaft having a proximal portion and a distal portion;
a therapeutic assembly at the distal portion of the elongated shaft and adapted to be located within a blood vessel of a human patient at a treatment site, the therapeutic assembly comprising an ultrasound transducer configured to transmit ultrasound waves; and
a balloon assembly at the distal portion of the elongated shaft and adjacent to the therapeutic assembly, wherein the balloon assembly comprises a first balloon and a second balloon circumferentially offset from the first balloon, and wherein the first and second balloons are configured to vary between a delivery configuration and a deployed configuration,
wherein, when the first and second balloons are in the deployed configuration, the balloon assembly is configured to selectively position the ultrasound transducer at a desired location within the blood vessel such that the ultrasound waves from the ultrasound transducer are focused on neural fibers within the blood vessel wall to induce thermal neuromodulation.

2. The ultrasound apparatus of claim 1 wherein the desired location comprises a selected radial distance between a surface of the transducer and the neural fibers.

3. The ultrasound apparatus of claim 1 wherein:
the transducer comprises a first side and a second side opposite the first side; and
the first balloon is adjacent the first side of the transducer and the second balloon is adjacent the second side of the transducer.

4. The ultrasound apparatus of claim 1 wherein, when in the deployed configuration, the first balloon has a first volume and the second balloon has a second volume different than the first volume.

5. The ultrasound apparatus of claim 1 wherein, when in the deployed configuration, the first balloon and the second balloon have the same or approximately the same volume.

6. The ultrasound apparatus of claim 1 wherein the first and second balloons are sized and shaped to allow fluid flow within the blood vessel when in the deployed configuration.

7. The ultrasound apparatus of claim 1 wherein the balloon assembly is located proximal of the transducer assembly along the elongated shaft.

8. The ultrasound apparatus of claim 1 wherein the balloon assembly is positioned over at least a portion of the transducer assembly along the elongated shaft.

9. The ultrasound apparatus of claim 1 wherein the ultrasound transducer is curved.

10. The ultrasound apparatus of claim 1 wherein the ultrasound transducer is configured to transmit the ultrasound waves over less than a full 360° about the blood vessel.

11. The ultrasound apparatus of claim 1 wherein the therapeutic assembly is configured to remain out of contact with a wall of the blood vessel during transmission of the ultrasound waves to the target depth.

12. The ultrasound apparatus of claim 1 wherein the elongated tubular shaft and the therapeutic assembly together define therethrough a guide wire lumen configured to slidably receive a medical guide wire, and wherein the therapeutic assembly is configured to be delivered via guide wire to the treatment site within the blood vessel.

13. The ultrasound apparatus of claim 1 wherein the therapeutic assembly is adapted to be located at a treatment site within a renal artery of the patient, and wherein the ultrasound transducer is configured to deliver the ultrasound waves to renal nerves along the renal artery.

14. A catheter apparatus, comprising:
an elongated shaft having a proximal portion and a distal portion; and
an ultrasound transducer at the distal portion of the elongated shaft and configured for intravascular placement within a renal blood vessel of a human patient, wherein the ultrasound transducer is configured to transmit ultrasound waves to renal nerves along the renal blood vessel to achieve thermal renal neuromodulation via heating;
a first balloon assembly at the distal portion of the elongated shaft and between the ultrasound transducer and a distal end of the elongated shaft; and
a second balloon assembly at the distal portion of the elongated shaft and located proximally of the ultrasound transducer along the elongated shaft,
wherein the first balloon assembly and the second balloon assembly are configured to vary between a low-profile delivery state and an expanded deployed state,
wherein, when the first balloon assembly and the second balloon assembly are in the deployed state, the first balloon assembly and the second balloon assembly are sized and shaped to position the ultrasound transducer within the renal blood vessel at a desired focal length from the renal nerves and out of contact with an interior wall of the renal blood vessel, and
wherein, when the first balloon assembly and the second balloon assembly are in the deployed state, the first balloon assembly and the second balloon assembly are sized and shaped to allow blood flow through the renal blood vessel.

15. The catheter apparatus of claim 15 wherein the distal portion is adapted to be located within a renal artery of the patient, and wherein the ultrasound transducer is configured to deliver the ultrasound waves to renal nerves along the renal artery.

16. The catheter apparatus of claim 15 wherein:
the first balloon assembly further comprises a first plurality of balloons, wherein each of the first plurality of balloons is circumferentially offset from one another about the elongated shaft; and
the second balloon assembly further comprises a second plurality of balloons, wherein each of the second plurality of balloons is circumferentially offset from one another about the elongated shaft.

17. A method, comprising:
positioning a distal portion of a catheter at a treatment site within or otherwise proximate to a vessel having a neural fiber proximate a wall of the vessel, wherein the distal portion includes—
a therapeutic assembly having an ultrasound transducer with a focal length, and
a balloon assembly having a first balloon and a second balloon circumferentially offset from the first balloon;
expanding the first balloon to a first volume;
expanding the second balloon to a second volume such that the transducer and the neural fiber are separated by the focal length; and
activating the therapeutic assembly to modulate the neural fiber.

18. The method of claim 18 wherein the first volume and the second volume are different.

19. The method of claim 18 wherein expanding the second balloon further comprises bringing the transducer closer to a portion of the wall of the vessel proximate to the neural fiber.

20. The method of claim 18 wherein activating the therapeutic assembly occurs at a first time, and wherein the method further comprises:
decreasing the first and/or second volumes to deflate the first and/or second balloons, respectively;
rotating the therapeutic assembly about a longitudinal axis of the shaft;
reinflating the first balloon and the second balloon such that the transducer and the neural fiber are separated by the focal length; and
activating the therapeutic assembly to modulate nerves of the patient at a second time.

21. The method of claim 18 wherein:
the neural fiber is a first neural fiber, and
activating the therapeutic assembly occurs at a first time; and
wherein the method further comprises—
decreasing the first and/or second volumes to deflate the first and/or second balloons, respectively;
rotating the therapeutic assembly about a longitudinal axis of the shaft;
reinflating the first balloon and the second balloon such that the transducer and a second neural fiber are separated by the focal length, wherein the second neural fiber is different than the first neural fiber; and
activating the therapeutic assembly to modulate nerves of the patient at a second time.

22. A method, comprising:
positioning a distal portion of an intravascular catheter at a first treatment site within or otherwise proximate to a vessel of a human patient, wherein the first vessel at the treatment site has a first diameter, and wherein the distal portion includes—
a therapeutic assembly configured to deliver ultrasound energy to neural fibers proximate a wall of the vessel, and
a balloon assembly having a first balloon and a second balloon;
expanding the first balloon to a first balloon first volume and the second balloon to a second balloon first volume, wherein the first balloon first volume is the same or different than the second balloon first volume;
activating the therapeutic assembly to modulate nerves of the patient;
removing the therapeutic assembly from the first treatment site;
positioning the distal portion at a second treatment site having a second diameter different than the first diameter;
expanding the first balloon to a first balloon second volume, that is the same or different than the first balloon first volume;
expanding the second balloon to a second balloon second volume that is the same or different than the first balloon first volume; and
activating the therapeutic assembly to modulate nerves of the patient at a second time.

23. The method of claim 22 wherein the first treatment site and the second treatment site comprise different portions of the vessel.

24. The method of claim 22 wherein the vessel is a first vessel, and wherein the first treatment site is located at the first vessel, and the second treatment site is located at a second vessel different than the first vessel.
